(19) United States
(12) Patent Application Publication
(10) Pub. No.: US 2018/0258375 A1
(43) Pub. Date: Sep. 13, 2018

(54) USE OF A LYOCELL FIBER

(71) Applicant: LENZING AKTIENGESELLSCHAFT, Lenzing (AT)

(72) Inventors: Martina OPIETNIK, 4861 Schörfling a Attersee (AT); Gisela GOLDHALM, 3363 Neufurth (AT); Heinrich FIRGO, 4840 Vöcklabruck (AT)

(21) Appl. No.: 15/760,369

(22) PCT Filed: Sep. 13, 2016
(86) PCT No.: PCT/EP2016/071486

§ 371 Date: Mar. 15, 2018

(30) Foreign Application Priority Data
Sep. 16, 2015 (EP) 15185393.4

(51) Int. Cl. C11D 17/04; C11D 1/62; A61K 8/02; A61K 8/41; A61K 8/73; A61K 8/27; A61K 8/19; A61Q 17/00; A61Q 19/10; C11D 3/12; C11D 3/22

(52) U.S. Cl. CPC C11D 17/049; C11D 1/62; A61K 8/0208; A61K 8/027; A61K 8/416; A61K 8/731; C11D 3/222; A61K 8/19; A61Q 17/005; A61Q 19/10; C11D 3/1233; C11D 3/1213; A61K 8/27

(57) ABSTRACT

The present invention relates to the use of a lyocell fiber in a product comprising a non-woven substrate and a liquid containing a compound with a quaternary ammonium salt, wherein said lyocell fiber is contained in said non-woven substrate, and wherein said lyocell fiber contains an essentially water-insoluble salt incorporated in said fiber.

### BACKGROUND OF THE INVENTION

### Field of the Invention

The present invention relates to the use of a lyocell fiber in a product comprising a non-woven substrate and a liquid containing a compound with a quaternary ammonium salt. Such products are in the following also referred to as “wet or moist wipes”.

Especially, the present invention relates to the use of a lyocell fiber in wet or moist wipes for hygiene products, especially for disinfecting and sanitizing wipes for home, food service, medical and other market areas.

For hygiene purposes wet or moist wipes, respectively, containing quaternary ammonium salts as active substances are commonly used. The wipes mainly consist of a non-woven substrate which is impregnated with a liquid, such as a solution or dispersion containing said quaternary ammonium salt. This liquid is in the following also referred to as “lotion”. The quaternary ammonium salt exerts an antimicrobial activity.

In the US, the compounds and the amounts of active ingredient present in an antimicrobial wipe solution are specified in EPA docket numbers: EPA-HQ-OPP-2006-0339 and EPA-HQ-OPP-2006-0338 and 21 code of Federal Regulations § 178.1010 (b) and (c). The FDA regulation applies to “food processing equipment and on other food-contact article as specified”, whereas the EPA regulation covers most of the other uses, primarily sanitation wipes for use with hard surfaces. § 178.1010 (b) specifies the compounds which can be used and § 178.1010 (c) specifies the amounts of the active ingredients in the solution in at least amounts and not to exceed amounts, whereas the EPA regulations specify the maximum quaternary ammonium salt concentrations.

Wet wipes which fulfill the regulations cited today mainly consist of Polyester (PES) fibers, Polypropylene (PP) fibers or mixtures thereof, as well as mixtures of these fibers with a low amount of only very few percents of cellulosic fibers.

Wet wipes are normally packed in packages with stacks of typically 10 to over 100 single pieces. During storage the lotion tends to seep from top to bottom due to gravity and poor interaction with the non-cellulosic fibers. This leads to a non-uniform distribution of the lotion which affects the effectiveness of a single wipe from the pack. Due to the hydrophobic properties of PES and PP fibers this effect is increased if aqueous formulations are employed.

To decrease this effect, cellulosic fibers may be used, as these fibers are hydrophilic and are able to improve the distribution of lotions. However, each cellulosic fiber contains anionic carboxylic groups. An interaction between the anionic carboxylic groups and the cationic substance (active substance) in the lotion reduces the effectiveness of these products.

To cope with this problem, the load of quaternary ammonium salts on the wipe can be increased in order to compensate for the chemical tie-up. However, this over-loading sometimes puts the product outside of the FDA monograph.

According to US 2011/0220311, dissolved metal salts are added to the lotion to prevent adsorption of quaternary ammonium salts by the cellulosic fibers. The salts have much smaller cations compared to the active substances in the lotion. Therefore, these cations can bind much faster to the surface of the cellulose and mask the anionic charges. As a consequence, the active substance does not bind to the cellulose and is free to work as a disinfectant.

According to US 2006/0193990, a binder compromising a polymer and a surfactant is added, which compensates the surface charge on the fibers of the web product, whereby the cationic functional agent remains free in the lotion and is deliverable to the surface in sufficient amounts for the desired disinfecting effect.

US2005/0245151 concerns a method for increasing the release of a cationic component of a lotion from a non-woven wet wipe material by adding a chemical blocking material (polyamide-epichlorhydrine resin, polyamide resin, melamine resin, high molecular weight cationic chemical material) to the non-woven fabric prior to saturation of the fabric with a lotion.

In WO 2007/016579 a cationic fibrous sanitizing substrate comprising a cationic fibrous compound, such as a chitosan fiber with a cationic finish, is disclosed.

U.S. Pat. No. 7,637,271 (The Clorox Company) discloses the addition of 0, 1-20% of a polyaluminium compound (chlorid, chlorohydrate, sulfate) to a cleaning composition.

According to the state of the art additional chemicals and after-treatment steps are necessary in order to influence the surface charge of the cellulosic fibers employed in wet and moist wipe products. This causes additional costs and chemical load.

There is a demand for wet wipes having sufficient release properties and to eliminate fluid migration in the package.

This problem underlying the present invention is solved by the use of a lyocell fiber in a product comprising a non-woven substrate and a liquid containing a compound with a quaternary ammonium salt, wherein said lyocell fiber is contained in said non-woven substrate, and wherein said lyocell fiber contains an essentially water-insoluble salt incorporated in said fiber.

Preferred embodiments of the present invention are disclosed in the dependent claims.

### DETAILED DESCRIPTION OF THE INVENTION

The use of a lyocell fiber containing an essentially water-insoluble salt incorporated in said fiber surprisingly solves the problem of wet or moist wipes discussed above.

Especially, by way of incorporating an essentially water-insoluble salt into the lyocell fiber, it has been found that there is no need for a further treatment or addition of further materials in the stage of producing the wet or moist wipe in order to avoid the problems discussed above.

The term “lyocell” is the generic name allocated by BISFA (The International Bureau for the Standardization of man made fibers) for cellulose fibers which are produced by dissolving cellulose in an organic solvent without the formation of a derivative and extruding fibers from said solution by means of a dry-wet spinning process or a melt-blown process. In this regard, an organic solvent is understood to be a mixture of an organic chemical and water. At present, N-methyl-morpholine-N-oxide (NMMO) is used as an organic solvent on a commercial scale.

In said process, the solution of the cellulose is usually extruded by means of a forming tool, whereby it is moulded. Via an air gap, the moulded solution gets into a precipitation bath, where the moulded body is obtained by precipitating the solution. The molded body is washed and optionally dried after further treatment steps. A process for the production of lyocell fibers is described, for instance, in U.S. Pat. No. 4,246,221. Lyocell fibers are distinguished by a high tensile strength, a high wet-modulus and a high loop strength.

Under the term “incorporated in said fiber”, the skilled artisan understands that the essentially water-insoluble salt is distributed within the cellulosic matrix of the fiber essentially throughout the whole cross-section of it.

It is well-known to modify cellulosic fibers and also lyocell fibers via the incorporation of various additives.

Incorporation of a modifying compound into a lyocell fiber can be achieved, as is known per se to the skilled artisan, by admixing the compound to the spinning solution before the spinning process and/or by admixing the compound to a precursor of the solution.

As a “precursor” of the spinning solution, starting and intermediate materials for the manufacture of the spinning solution are to be understood, especially
the starting cellulose material employed, e.g. pulp
the solvent employed (aqueous tertiary amine oxide, in the following the term “NMMO” is used as an abbreviation for all suitable amine oxides)
a mixture of the starting cellulose material with the solvent NMMO, especially a suspension of the cellulose in an aqueous NMMO, starting from which the solution can be made.

WO 2009/036481 discloses a lyocell fiber containing a material selected from the group consisting of pearl powder, ground nacre and mixtures thereof incorporated therein.

In the following, a lyocell fiber containing an essentially water-insoluble salt incorporated therein, as employed by the present invention, is referred to as the “modified lyocell fiber”.

Under an “essentially water-insoluble” salt the skilled artisan understands a salt which has a very low solubility in water so that it is not washed out in the aqueous milieu of producing lyocell fibers including their aftertreatment, or is only washed out to an extent which is acceptable in terms of the ecological and economical requirements of a commercial production.

Preferably, the salt employed in the present invention has a solubility in water at 15° C. to 25° C. of 5 g/l or less, such as 3 g/l or less, 1 g/l or less, especially preferred less than 0.1 g/l.

Preferably, the salt is soluble under acidic conditions.

Typically, the lotion containing the compound with a quaternary ammonium salt, which is applied to a non-woven substrate in order to produce a wet or moist wipe, has a pH-value of about of smaller than 7, especially of from 3 to 6, such as of from 4.0 to 5.5.

If under these acidic conditions the salt contained in the fiber is soluble, it will dissociate to a certain extent. The cation of the salt then binds to the cellulose, thus masking the anionic charges of the cellulose without the need of the addition of further additives.

As an alternative, the modified lyocell fiber, or the non-woven substrate containing the modified lyocell fiber, can be treated with an acidic medium before the treatment with the lotion containing the quaternary ammonium salts. Again by way of such treatment the anionic charges of the cellulosic fiber would be masked. This alternative would enable the use of lotions with no acidic pH-value in the production stage of the wet or moist wipe. Furthermore, more stringent treatment conditions (such as the use of stronger acids) can be applied under this alternative.

Lyocell fibers typically contain an amount of about 20 μmol to 50 μmol, especially 25 μmol to 40 μmol carboxylic, i.e. anionic groups per g of fiber. Generally spoken, therefore, the objective is to provide a modified fiber in which, upon an acidic treatment of the fiber, a sufficient amount of cations from the salt contained in the fiber will be released in order to mask these anionic charges as completely as possible.

As is readily apparent, this objective can be solved on the basis of the skilled artisan's knowledge, by appropriately selecting the parameters of
the nature of the salt and, thus, both its valence and its solubility
the amount of salt contained in the fiber and
the nature and amount of the acidic liquid with which the fibers and/or the non-woven substrate are treated.

In a preferred embodiment of the present invention, the essentially water-insoluble salt employed may be selected from the group consisting of salts of alkali metals, earth alkali metals, transition metals, aluminum and mixtures thereof.

Further preferably, the salt is an organic salt.

Especially preferred, the salt is selected from the group consisting of calcium carbonate, zinc oxide, calcium phosphate, magnesium hydroxide, aluminum hydroxide, calcium oxalate, barium carbonate, organic oxalates and mixtures thereof.

The lyocell fiber used according to the present invention preferably contains said an organic salt in an amount of from 0.1 wt % to 50 wt. %, preferably of from 1 wt. % to 10 wt. %, especially preferred of from 2 wt. % to 7 wt. %, based on the weight of the dry fiber.

The quaternary ammonium salt employed for making the wet or moist wipe is preferably a quaternary ammonium chloride.

Especially, said quaternary ammonium salt may be selected from the group consisting of n alkylbenzyldimethylammoniumchloride (BAC or ABDAC), di-n-alkyldimethyl ammoniumchloride (DDAC), n-alkyldimethylethylbenzylammonium chloride (ADEBAC), n-alkyltrimethylammonium chloride, octyldecyldimethylammonium chloride, didecyldimethylammonium chloride, dioctyldimethylammonium chloride, benzethonium chloride and mixtures thereof.

The non-woven substrate containing the modified lyocell fiber can be manufactured by means known to the skilled artisan. In a preferred embodiment of the present invention, the non-woven substrate containing the modified lyocell fiber is a melt-blown product. The manufacture of melt-blown products from solutions of cellulose in NMMO is known, e.g. from, inter alia, WO 98/26122, WO 99/47733, WO 98/07911, U.S. Pat. No. 6,197,230, WO 99/64649, WO 05/106085, EP 1 358 369 and WO 2007/124521.

Preferably, the non-woven substrate contains the modified lyocell fiber in an amount of from 5 wt. % to 100 wt. %, preferably 10 wt. % to 50 wt. %, especially preferred 20 wt. % to 30 wt. %, based on the weight of the dry substrate.

Under a non-woven substrate containing the modified lyocell fiber in an amount of “100 wt. %”, the skilled artisan understands a substrate that essentially consists of said modified lyocell fiber, such as a melt-blown non-woven product produced from a solution of cellulose in NMMO. The presence of minor amounts of additives, such as for surface treatment of the fiber and/or the non-woven substrate, is not excluded thereby.

As known per se, the non-woven substrate may contain a mixture of the modified lyocell fiber with hydrophobic fibers, such as PP fibers, PES fibers and mixtures thereof.

The present invention also relates to a product comprising a non-woven substrate and a solution containing a compound with a quaternary ammonium salt, wherein a lyocell fiber is contained in said non-woven substrate, and wherein said lyocell fiber contains an essentially water-insoluble salt incorporated in said fiber.

As to the details of the fiber employed in the product of the present invention, i.e. the wet or moist wipe, the nature of the salt and other preferred embodiments, reference may be made to the above paragraphs.

### EXAMPLES

### Example 1: Improvement of Release Properties of BAC by Incorporation of CaCO₃

Lyocell fibers having 1.1 wt %; 2.9 wt %; 4.1 wt %; 4.8 wt % and 6.2 wt % (based on bone-dry fiber), respectively, CaCO₃ incorporated therein were produced according to the state of the art. Thus, a 13 wt % spinning dope of cellulose in aqueous NMMO was prepared containing the above-named concentrations of CaCO3. Subsequent spinning of the dope using the lyocell process led to the final fiber (1.7 dtex, 38 mm).

These air-dried fibers were mingled with 400 wt % (based on air-dried fibers) aqueous BAC-solution (alkyldimethylbenzylammonium chloride, 1000 ppm), and the release properties were determined using the titration method explained below in example 4. The aqueous BAC solution was adjusted to an acidic pH.

It becomes apparent that with increased incorporation of CaCO₃ the concentration of accessible active substance increases. In table 1 the results of the titrations of the different CaCO₃ fiber types are summarized.

TABLE 1
Evaluation of titrations of the different CaCO3 incorporated fibers.

| | Release of |
|---|---|
| Fiber type | BAC [%] |
| 1.1% CaCO₃ | 75 |
| 2.9% CaCO₃ | 77 |
| 4.1% CaCO₃ | 80 |
| 4.8% CaCO₃ | 86 |
| 6.2% CaCO₃ | 90 |

### Example 2: Improvement of Release Properties of BAC by Incorporation of ZnO

Lyocell fibers having 1.5 wt %; 2 wt %; 2.5 wt % and 3.0 wt % (based on bone-dry fiber), respectively, ZnO incorporated therein were produced according to the state of the art. Thus, a 13 wt % spinning dope of cellulose in aqueous NMMO was prepared containing the above-named concentrations of ZnO. Subsequent spinning of the dope using the lyocell process led to the final fiber (1.7 dtex, 38 mm).

These air-dried fibers were mingled with 400 wt % (based on air-dried fibers) aqueous BAC-solution (alkyldimethylbenzylammonium chloride, 1000 ppm), and the release properties were determined using the titration method explained below in example 4. The aqueous BAC solution was adjusted to an acidic pH.

In table 2 the results of the titrations of the different ZnO fiber types are summarized.

TABLE 2
Evaluation of titrations of the different ZnO incorporated fibers.

| | Release of |
|---|---|
| Fiber type | BAC [%] |
| 1.5% ZnO | 85 |
| 2% ZnO | 90 |
| 2.5% ZnO | 90 |
| 3% ZnO | 92 |

### Example 3: At Least Twofold Increase of BAC Release Properties by Incorporation of Insoluble Salts

The fiber types mentioned in example 1 and 2 were compared with standard lyocell fibers not containing a water-insoluble salt regarding their BAC release properties.

It becomes apparent that the fiber types employed according to the present invention compared to the standard lyocell fibers show an at least twofold release of BAC.

The quaternary release was determined using the titration method described in example 4. In table 3, the results of the titrations of the different fiber types are summarized.

TABLE 3
Titration results of different fiber types.

| | Release of |
|---|---|
| Fiber type | BAC (%) |
| TENCEL ® standard | 29 |
| 4.8% CaCO₃ | 86 |
| 2% ZnO | 90 |

### Example 4: Determination of BAC Release Properties Using a 2-Phase-Titration

The cationic substance (BAC or other QACs) to be determined is titrated using sodium dodecylsulfate in a two-phase system containing water and a chlorinated solvent (dichloromethane, chloroform, . . . ) using bromophenol blue as indicator.

Cationic substances form a complex with bromophenyl blue which is soluble in the chlorinated solvent. Titration of this complex with the anionic surfactant leads to a replacement of the indicator anion and the new formed complex passes over to the aqueous phase. At the end of the titration the released indicator colors the chlorinated phase blue/violet, whereas the aqueous phase recolors yellowish/colorless.

### Example 5: Fiber Data of the Fibers with Improved QAC Release Properties

The fiber data of the fiber types described in examples 1 and 2 was determined. Table 4 shows that the fiber data does not change with the incorporation of the mentioned salts.

TABLE 4
Fiber data

| | | Cross | | |
|---|---|---|---|---|
| | Incorporation | section | FFk | FDk |
| | [%] | [dtex] | [cN/tex] | [%] |
| Standard lyocell | 0 | 1.7 | 30.5 | 12 |
| fiber | | | | |
| 4.1% CaCO₃ | 4.1 | 1.35 | 30.5 | 12.1 |
| 4.8% CaCO₃ | 4.8 | 1.84 | 28.8 | 12.9 |
| 6.2% CaCO₃ | 6.2 | 1.76 | 28 | 12.9 |
| 3% ZnO | 3 | 1.82 | 34.2 | 13.4 |
| 2.5% ZnO | 2.5 | 1.68 | 36.7 | 13 |
| 2% ZnO | 2 | 1.84 | 33 | 13.5 |
| 1.5% ZnO | 1.5 | 1.7 | 34.06 | 13.6 |

FFk: Tenacity conditioned
FDk: Elongation conditioned (FDk).

1-8. (canceled)

9. A product comprising a non-woven substrate and a solution comprising a compound with a quaternary ammonium salt, wherein said non-woven substrate comprises a lyocell fiber which comprises an essentially water-insoluble salt incorporated in said fiber.

10. The product according to claim 9, wherein said essentially water-insoluble salt has a solubility in water at 15° C. to 25° C. of 5 g/l or less.

11. The product according to claim 9, wherein said essentially water-insoluble salt is selected from the group consisting of salts of alkali metals, earth alkali metals, transition metals, aluminum and mixtures thereof.

12. The product according claim 9, wherein said essentially water-insoluble salt is an organic salt.

13. The product according to claim 9, wherein said essentially water-insoluble salt is selected from the group consisting of calcium carbonate, zinc oxide, calcium phosphate, magnesium hydroxide, aluminum hydroxide, calcium oxalate, barium carbonate, organic oxalates and mixtures thereof.

14. The product according to claim 9, wherein said lyocell fiber comprises said essentially water-insoluble salt in an amount of from 0.1 wt % to 50 wt. %, based on the weight of the dry fiber.

15. The product according to claim 9, wherein said non-woven substrate comprising said lyocell fiber is a melt-blown product.

16. The product according to claim 9, wherein said non-woven substrate comprises said lyocell fiber in an amount of from 5 wt. % to 100 wt. %, based on the weight of the dry substrate.

17. The product according to claim 10, wherein said essentially water-insoluble salt has a solubility in water at 15° C. to 25° C. of 3 g/l or less.

18. The product according to claim 17, wherein said essentially water-insoluble salt has a solubility in water at 15° C. to 25° C. of 1 g/l or less.

19. The product according to claim 18, wherein said essentially water-insoluble salt has a solubility in water at 15° C. to 25° C. of less than 0.1 g/l.

20. The product according to claim 14, wherein said lyocell fiber comprises said essentially water-insoluble salt in an amount of from 1 wt. % to 10 wt. %, based on the weight of the dry fiber.

21. The product according to claim 20, wherein said lyocell fiber comprises said essentially water-insoluble salt in an amount of from 2 wt. % to 7 wt. %, based on the weight of the dry fiber.

22. The product according to claim 16, wherein said non-woven substrate comprises said lyocell fiber in an amount of from 10 wt. % to 80 wt. %, based on the weight of the dry substrate.

23. The product according to claim 22, wherein said non-woven substrate comprises said lyocell fiber in an amount of from 20 wt. % to 60 wt. %, based on the weight of the dry substrate.

24. The product according to claim 23, wherein said non-woven substrate comprises said lyocell fiber in an amount of from 20 wt. % to 30 wt. %, based on the weight of the dry substrate.
